(19) United States
(12) Patent Application Publication
(10) Pub. No.: US 2025/0232033 A1
(43) Pub. Date: Jul. 17, 2025

(54) THRESHOLD SIGNATURE BASED MEDICAL DEVICE MANAGEMENT

(71) Applicant: ICU Medical, Inc., San Clemente, CA (US)

(72) Inventors: S. Sree Vivek, Chennai (IN); Hrishikesh Anil Dandekar, Pune (IN); Chaitanya Mattur Srinivasamurthy, Lake Forest, IL (US)

(21) Appl. No.: 18/929,308

(22) Filed: Oct. 28, 2024

Related U.S. Application Data
(63) Continuation of application No. 16/869,404, filed on May 7, 2020, now Pat. No. 12,130,910.
Application No. 16/869,404 is a continuation of application No. PCT/US2020/031664, filed on May 6, 2020.
(60) Provisional application No. 62/845,115, filed on May 8, 2019.

(51) Int. Cl. G06F 21/55; H04L 9/08; H04L 9/32

(52) U.S. Cl. CPC G06F 21/554; H04L 9/085; H04L 9/0894; H04L 9/3247; H04L 2209/16; H04L 2209/88

(57) ABSTRACT

The present disclosure is directed to managing device authorization through the use of digital signature thresholds. Individual components of a device, or individual devices in a network environment, are associated with separate secret shares from which a digital signature can be derived. The digital signature may be used to authorize performance of a function. A threshold number of such secret shares are used in order to derive the digital signature. Therefore, an authorization process that relies on digital signature verification to determine that a function is authorized will do so if a threshold number of secret shares are available at authorization time.

### CROSS-REFERENCE TO RELATED APPLICATIONS

The present application is a continuation of U.S. patent application Ser. No. 16/869,404, filed on May 7, 2020 and title “Threshold Signature Based Medical Device Management,” which is a continuation of International Patent Application No. PCT/US2020/031664, filed on May 6, 2020 and titled “Threshold Signature Based Medical Device Management,” which claims priority to U.S. Provisional Patent Application No. 62/845,115, filed on May 8, 2019 and titled “Threshold Signature Based Medical Device Management,” the contents of both of which are incorporated by reference herein.

### TECHNICAL FIELD

This disclosure relates to the field of medical device management, and particularly to systems and methods for secure authorization of medical devices.

### BACKGROUND

Computing systems, such as medical devices that have processors and other computing components, execute software that controls the functions performed by the computing systems. Software is typically stored in a persistent data store (e.g., hard disk, flash memory, etc.), and loaded into volatile memory (e.g., random access memory or RAM) for execution. Computing systems often verify whether software is authorized prior to execution. For example, the computing system may determine whether a license has been obtained, authorizing execution of the software. If a valid license has been obtained, then execution of the software may be permitted. If no license has been obtained, then execution of the software may be blocked.

### SUMMARY

Various techniques for managing the operation of devices using threshold signature-based authorization are described herein. These techniques may include creating shares of digital signature generation keys, and assigning the shares to devices or individual components of devices. The shares may then be used to authorize performance of particular functions so long as a threshold number of shares—or a threshold amount of weighted shares—are available during the authorization procedure. For example, threshold signature-based authorization may be used to validate licenses, validate the presence of certain components, respond to security events, ensure devices are being used in the intended environment, etc. These and other embodiments are described in greater detail below with reference to FIGS. 1-6. Although many of the examples are described in the context of medical devices, functions, and environments (including infusion pumps, medication dispensing functions, and hospital or clinical environments), the techniques described herein can be applied to other types of devices, functions, and environments.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of an example network environment including a device management system and various devices according to some embodiments.

FIG. 2 is a block diagram illustrating data flows and interactions between a device and a device management system during pre-authorization setup according to some embodiments.

FIG. 3 is a flow diagram of an illustrative process for threshold signature authorization of a device according to some embodiments.

FIG. 4 is a block diagram illustrating data flows and processing performed by a device during weighted authorization according to some embodiments.

FIG. 5 is a block diagram illustrating data flows and processing performed during group-based authorization according to some embodiments.

FIG. 6 is a block diagram illustrating data flows and processing performed by a device during dynamic weighted authorization according to some embodiments.

### DETAILED DESCRIPTION OF ILLUSTRATIVE EMBODIMENTS

The present disclosure is directed to managing device authorization through the use of digital signature thresholds. Individual components of a device, or individual devices in a network environment, are associated with separate shares of data from which a digital signature can be generated. For example, the shares of data may be used to generate shares of a digital signature, and the digital signature may be used to authorize performance of a function. The shares of data may be referred to as “secret shares,” or simply as “shares” for convenience. In an illustrative embodiment, a threshold number of secret shares are required in order to perform any private operation such as generating a digital signature, recovering secret data, etc. Therefore, an authorization process that relies on digital signature verification to determine that a function is authorized will only do so if a threshold number of secret shares are available at authorization time. If fewer than the threshold number of secret shares are available, then the requested function (or operation of the device as a whole) is blocked. In this way, a device can be required to maintain a threshold number of original or authorized components to continue using licensed software or to continue executing at all. In a similar manner, a device can be prohibited from operation unless a threshold number of connected devices participate in an authorization procedure, or a threshold number of components of the device participate in an authorization procedure.

Some aspects of the present disclosure relate to generating secret shares for individual components of a medical device (e.g., an infusion pump), and using the secret shares to determine whether to authorize a function of the medical device. In some embodiments, software may be installed on a medical device, and a valid license may be required in order to execute the software. During installation or initial licensing, the license may be tied to the current configuration of the medical device such that the software is only permitted to execute if at least a threshold number of components of the medical device remain the same. To enforce this licensing requirement, a different secret share can be assigned to various individual components of the device. For example, there may be n secret shares of such data generated, one for each of n different components of the device. When the software is to execute, an authorization procedure can be performed. An illustrative authorization procedure may involve: (1) generating a different portion or “signature share” of a digital signature using the same message and each of the secret shares associated with device components that are still present; (2) generating a digital signature using the signature shares; and (3) verifying the authenticity of the digital signature (e.g., verifying that the digital signature was generated using a threshold number of secret shares and the message). However, a valid digital signature may only be generated if there are at least a threshold number t of secret shares available at authorization time, where 0<t<n. If too many components have changed or been removed, then fewer than t secret shares may be obtained, a valid digital signature may not be generated, and the verification of the digital signature will fail. In this way, a medical device may be prevented from executing the software (or otherwise performing a now-unauthorized function) if the software has been copied onto a different device, or if some other event has occurred (e.g., the software may no longer be compatible with the medical device in the device's current configuration).

Additional aspects of the present disclosure relate to treating some device components differently than other device components during an authorization process. The mechanism by which the components are treated differently may be the use of weighting factors that are assigned to the components. The weighting factors may also be referred to simply as “weights” for convenience. In some embodiments, a particular component or subset of components may be determined to be more important than other components. The more important components may be assigned a higher weight than less important components. When an authorization process is subsequently performed, the various secret shares and weights associated with the device components currently available to the medical device may be obtained. In generating a digital signature from the weights and secret shares, a threshold amount of weighted secret shares may be required in order to generate a valid digital signature. For example, if relatively important component is not present but all other components are present, the authorization procedure will fail. However, if a comparatively less important component is not present but all other components are present, the authorization procedure may complete successfully. In this way, a medical device may be prevented from operating with too many replacements of critical components, critical replacement components that are not of the correct type, too few original or authorized versions of critical components, etc. Such limitations can improve safety and reliability. In some embodiments, the different components may be assigned different numbers of shares, rather than weights with different values, to indicate the relative importance of the components.

Further aspects of the present disclosure relate to group-based authorization of individual medical devices in a network environment. Group-based authorization may also be referred to as “swarm authentication.” In some embodiments, a medical device that is attempting to perform a particular function (e.g., connect to a network server or dispense medication) may be prohibited from doing so unless a threshold number of other medical devices are able to participate in the authorization. This can prevent malicious or otherwise undesirable use of a medical device that may be lost or stolen, because the medical device may not be operating in a network environment (e.g., a hospital or clinic) in which a threshold number of other devices are available to participate in the authorization procedure. For example, each medical device may be provided with or otherwise assigned a secret share. When a particular device attempts to perform a function that requires authorization, a network server can determine which other devices are connected to the network server, and obtain or otherwise access the secret shares for each of the available devices. If a threshold number of secret shares are available, a valid digital signature may be successfully generated, the digital signature may be successfully verified, and the device may then be authorized to perform the function. Otherwise, the device may be blocked from performing the function. Therefore, a medical device that has become lost or stolen may be prevented from operating because it may not be able to connect to the correct clinical network and the group-based authorization process may not be able to obtain the threshold number of secret shares.

Still further aspects of the present disclosure relate to system-wide participation in security authorization. A multi-component system, such as a medical device, may implement dynamically-weighted threshold-based authorization to authorize performance of particular functions (or operation of the device at all). For example, individual components of system may be assigned a starting weight or number of secret shares. As an individual component detects security events (e.g., invalid login attempts, system security override attempts, etc.), the component can dynamically lower the weight that it contributes to system-wide authorization. Other components may perform in a similar manner. When the system attempts to authorize performance of a particular function, a threshold number of weighted secret shares may be required in order to generate a valid digital signature and successfully authorize the function. If a threshold amount of weighted secret shares are not available due to dynamically lowered weights, the authorization process will fail. In this way, individual components of a system can actively participate in the authorization determinations made by the system, even if some components do not otherwise perform security functions.

Although aspects of some embodiments described in the disclosure will focus, for the purpose of illustration, on particular examples of devices, cryptographic secrets, threshold-based authorization algorithms, and the like, the examples are illustrative only and are not intended to be limiting. In some embodiments, the systems and methods described herein may be applied to additional or alternative devices, cryptographic secrets, threshold-based authorization algorithms, etc. Various aspects of the disclosure will now be described with regard to certain examples and embodiments, which are intended to illustrate but not limit the disclosure.

### Overview of Example Network Environment

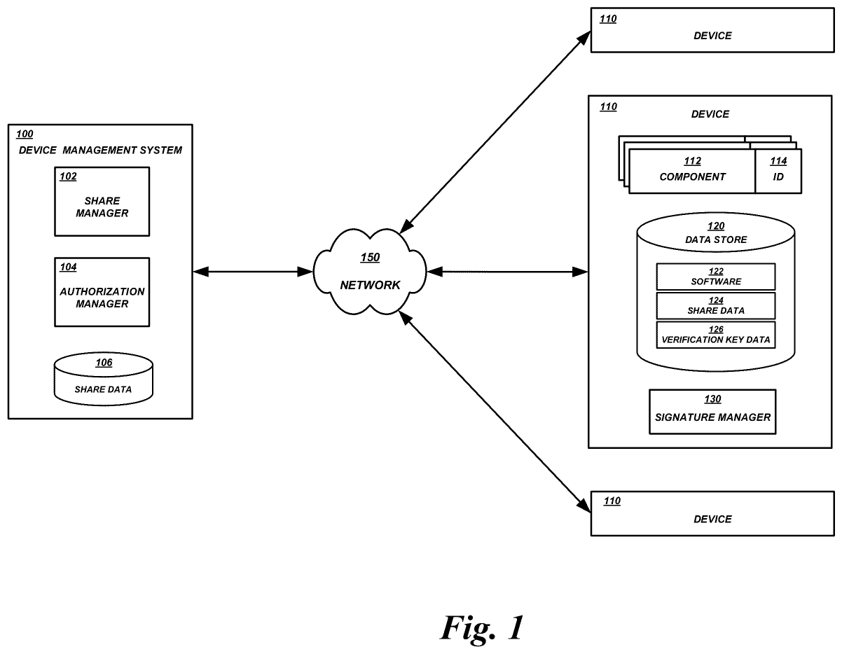

FIG. 1 illustrates network environment in which a device management system 100 communicates with one or more devices 110 via a communication network 150. Illustratively, the environment may include one or more healthcare facilities (e.g., hospitals) in which the devices 110 are medical devices and the device management system 100 is (or is part of) a locally-hosted or cloud-based system to manage use of the medical devices.

In some embodiments, a communication network 150 (also referred to simply as a “network”) may be a publicly-accessible network of linked networks, possibly operated by various distinct parties, such as the Internet. In some cases, the network 150 may be or include a private network, personal area network, local area network, wide area network, global area network, cable network, satellite network, cellular data network, etc., or a combination thereof, some or all of which may or may not have access to and/or from the Internet.

The device management system 100 may be a logical association of one or more computing devices for managing the generation and distribution of shares, performance of authorization procedures, and the like. For example, the device management system 100 can include one or more share managers 102. A share manager 102 may correspond to one or more server computing devices for generating secret shares and distributing the secret shares to the various devices 110 that use them. In some embodiments, the secret shares may also or alternatively be stored in a data store 106 corresponding to one or more data storage devices. The device management system 100 may also include one or more authorization managers 104. An authorization manager 104 may correspond to one or more computing devices configured to perform authorization procedures using the secret shares generated by the share manager 102.

In some embodiments, the features and services provided by the device management system 100 may be implemented as web services consumable via one or more communication networks. In further embodiments, the device management system 100 and/or individual components thereof may be provided by one more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment.

The devices 110 may correspond to any of a variety of devices that utilize or are subject to threshold-based authorization. Individual devices 110 may include various components 112, such as processors, network interface cards, volatile memory, long term storage, displays, and the like. Some or all of the components 112 may be associated with corresponding identifiers 114. For example, a processor component 112 may have a unique serial number that serves as the corresponding identifier 114. A memory component 112 may be associated with a different unique serial number that services as the corresponding identifier 114. A long-term storage component 112 (such as a disk drive) may be associated with its own unique serial number that serves as the corresponding identifier 114. In some embodiments, a component with a corresponding identifier may be a software component. For example, an operating system 122 may be stored in a data store 120 (such as a disk drive). The operating system 122 may be associated with its own unique identifier, as with the other components 112, and may therefore be treated similar to the other components 112 (e.g., assigned shares, weights, etc.). The data store 120 may be associated with its own unique identifier, as with the other components 112.

The devices 110 may also include components for performing secret share processing and/or authorization processes. In some embodiments, a device 110 may include a signature manager 130. The signature manager 130 may be a hardware-based component, or a software-based application or subsystem that runs on a processor of the device 110. The signature manager 130 may perform certain secret share processing and authorization processes, as described in greater detail below. For example, the signature manager 130 may access share data 124 and a verification key data 126 from the data store 120 for use in secret share processing and authorization processes. In some embodiments, the share data 124 and/or verification key data 126 may be stored in signature manager 130 or in some other secure location separate from the data store 120.

In some embodiments, the devices 110 may be or include medical devices, such as infusion pumps, with various components 112. For example, an infusion pump may include a motor controller unit (“MCU”) configured to control the motor that dispenses medication, a communications engine (“CE”) configured to manage network communications to/from the pump, operational software, and various other infusion pump-specific or medical-device specific components, any or all of which may be associated with respective identifiers 114. In addition, the devices 110 themselves may include different types of certain devices, such as different types of infusion pumps. Each type of infusion pump and even different versions of the same type of infusion pump may operate with a different operating system, use a different type of MCU and/or CE, etc.

### Secret Share Generation and Distribution Process

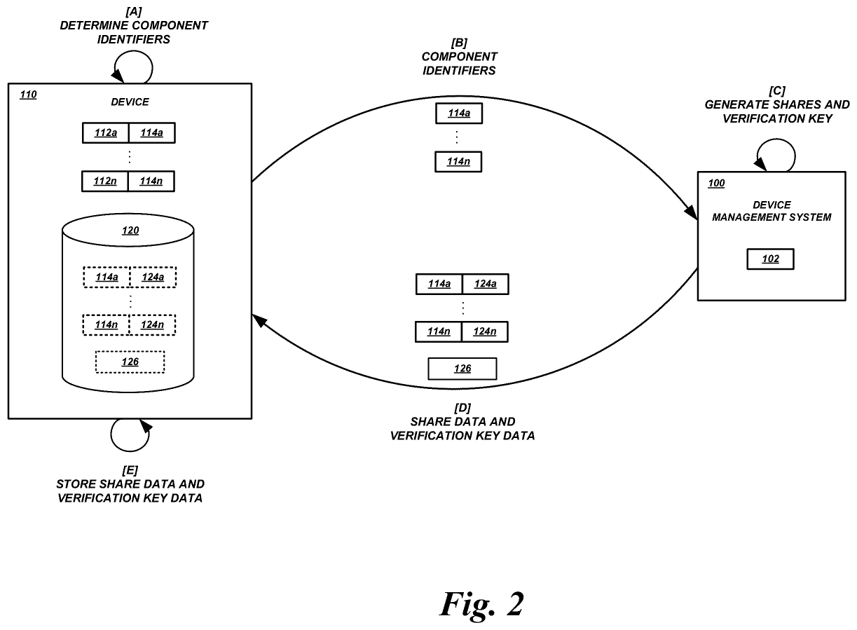

FIG. 2 is a block diagram of illustrative data flows and interactions between a device 110 and a device management system 100 during pre-authorization setup. The displayed interactions may occur during installation or initial licensing of software (or some other component). For example, software may be installed on the device 110. In order verify that the software is licensed for use on the device 110, the software may perform (or cause to be performed) an authorization process that only authorizes execution of the software if a threshold number of components 112a . . . 112n that were present on the device 110 during software installation are still present on the device. In order to facilitate this authorization process, a set of secret shares for digital signature generation may be obtained, assigned to the components 112a . . . 112n, and stored on the device 110. In some embodiments, the secret shares may each be a private encryption key or other key for use with a cryptographic function.

At [A], the device 110 can determine identifiers for a set of n components of the device 110. The value of n may depend on the software being installed, the number of components 112 present in the device 110, requirements of the software, etc. For example, n may be 4, and the set of n components may include a processor, memory, network interface, and battery. Each of the components may have an identifier with which they are already associated (e.g., a serial number). In some embodiments, components may be assigned identifiers as part of the pre-authorization setup. The identifiers may be globally-unique identifiers, or identifiers that are unique for at least the type of component to which they are assigned (e.g., identifiers that, in combination with other data such as model numbers, provide substantially unique identification of the respective components).

At [B], the device 110 can send the identifiers the device management system 100. For example, the device 110 may transmit the identifiers serially, asynchronously, or in a batch to the device management system 100.

At [C], the share manager 102 or some other module or component of the device management system 100 generates secret shares, such as individual digital signature share generation keys. A different secret share may be generated for each of the n component identifiers that have been received. The secret shares may be generated such that at least a threshold number t (where n>=t>0) of the secret shares must be available in order to generate a digital signature and proceed with an authorization process. The share manger 102 may also generate a verification key that is used during the authorization process to verify a digital signature generated using signature shares that have been generated using the secret shares.

In some embodiments, secret shares may be generated using Lagrange interpolation and Shamir's secret sharing to determine a random polynomial function from which the secret shares are generated. The different secret shares may correspond to values of the polynomial function for different input values (e.g., x₁, x₂, . . . , xₙ). The digital signature generation key may correspond to a value of the polynomial function for a particular predetermined input value (e.g., x=0). At authorization time, if at least t secret shares are available (corresponding to at least t different values of the polynomial function for t different input values), then the polynomial function itself may be derived from the secret shares without any other prior knowledge of the polynomial function. The polynomial function may then be evaluated for the predetermined input value that is used to determine the digital signature generation key. If the digital signature generation key is correct, then a digital signature generated using the key will be verified using the verification key that is generated by the share manager 102. If at least t different secret shares are not available, then it may be impossible or computationally infeasible to determine the polynomial function or to otherwise derive the correct digital signature generation key. For example, if there are t−1 shares available, then there may be an infinite number of possible polynomial functions that could be derived from the shares.

The example generation of secret shares described herein is illustrative only, and is not intended to be exhaustive or limiting. In some embodiments, other methods for generating secret shares may be used, such as verifiable secret sharing, Blakely's secret sharing, and the like. Example secret sharing schemes are described in “Secret-Sharing Schemes: A Survey” by Amos Beimel, and “How to Share a Secret” by Adi Shamir, the contents of both of which are incorporated by reference herein and made part of this specification.

At [D], the share manager 102 or some other module or component of the device management system 100 can send share data representing the secret shares, and verification key data representing the verification key, to the device 110. For example, the share manager 102 may transmit the share data and verification key data serially, asynchronously, or in a batch to the device 110.

At [E], the device 110 can store the share data and the verification key data. For example, share data 124a . . . 124n representing secret shares associated with corresponding identifiers 114a . . . 114n, and verification key data 126 representing the verification key, may be stored in the data store 120. In some embodiments, the signature manager 130 may store the share data 124 and verification key data 126 in a secure manner. For example, the share data 124 may be stored such that it is inaccessible outside of the signature manager 130, or such that individual shares are only accessible by querying the signature manager 130 with a valid identifier.

Once the secret shares and verification key have been generated and stored, the device 110 may be ready to execute an authorization process to authorize execution of a particular function such as execution of licensed software.

### Threshold-Based Authorization Process

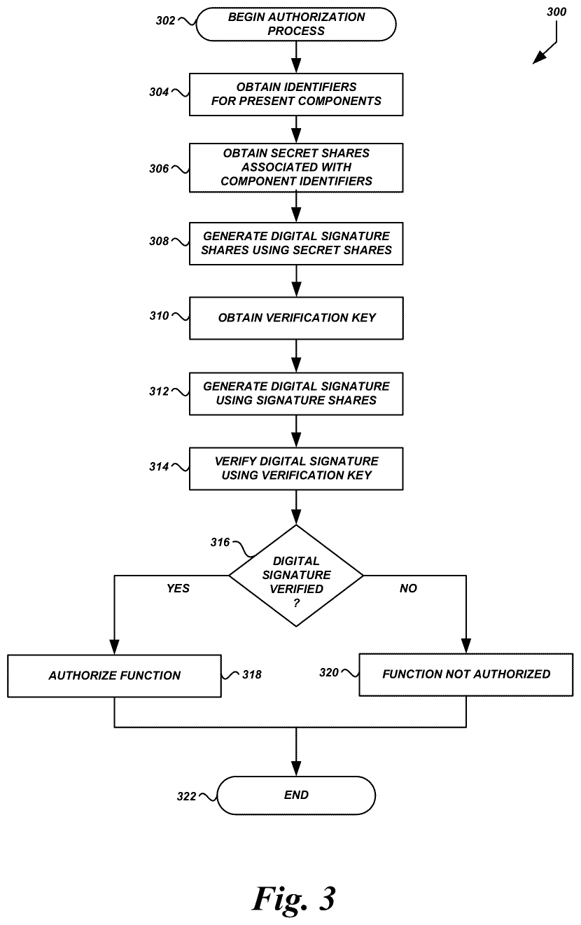

FIG. 3 is a flow diagram of an illustrative process 300 that may be executed by a signature manager 130 or some other component to determine whether a particular function is authorized. Advantageously, the process 300 can authorize the function if a threshold number t of secret shares are able to be obtained, corresponding to a threshold number t of components that were part of the device 110 when the pre-authorization setup process was performed. Otherwise, the process 300 cannot authorize the function if a threshold number t of secret shares are not able to be obtained. In this way, the authorization process can ensure that an authorized function is tied to a threshold number of components, and that the function (e.g., execution of software) will not be authorized if the software is installed on a different device 110, if too many original components 112 have been replace or removed, etc.

The process 300 shown in FIG. 3 begins at block 302. The process 300 may begin in response to an event, such as when a software application begins execution, or some other operation is to be performed. When the process 300 is initiated, a set of executable program instructions stored on one or more non-transitory computer-readable media (e.g., hard drive, flash memory, removable media, etc.) may be loaded into memory (e.g., random access memory or “RAM”) of the device 110. The executable instructions may then be executed by a hardware-based computer processor (e.g., a central processing unit or “CPU”) of the computing device. In some embodiments, the process 300 or portions thereof may be implemented on multiple processors, serially or in parallel.

At block 304, the signature manager 130 or some other module or component of the device 110 can obtain identifiers for the components 112 that are present in the device 110. In some embodiments, the signature manager 130 can obtain identifiers 114 for all components 112 of the device 110, or for only certain components 112 (e.g., the n most important components, or the components specified for the function being authorized).

At block 306, the signature manager 130 or some other module or component of the device 110 can obtain secret shares for the identifiers obtained at block 304. In some embodiments the signature manager 130 can query the data store 120 for share data 124 representing the secret shares that correspond to the obtained identifiers 114. There may be more secret shares in the data store 120 than there are identifiers (e.g., if a component 112 was removed but not replaced), there may be more identifiers than secret shares in the data store 120 (e.g., if a component 112 was added without another component being removed), or there may be the same number of secret shares as components (e.g., no components have been added or removed at all or none without the same number of components being removed or added, respectively).

At block 308, the signature manager 130 or some other module or component of the device 110 can generate digital signature shares, also referred to simply as “signature shares,” using the secret shares retrieved above at block 306. In some embodiments, the signature manager 130 may generate a separate signature share using each secret share and the same data item. For example, a predefined encryption function may use a secret share to encrypt a data item, such as a message or some other token. The token may be a randomly-generated value, a token provided to the authorization process, or some other input that is to serve as the unencrypted message to be digitally signed. The token may be encrypted directly, or it may be processed first (e.g., a hash may be generated using a hashing algorithm). The output of the encryption function may be an encrypted version of the input value (or hashed input value). Illustratively, the signature manager 130 may use a first secret share to encrypt a token and generate a first signature share. The signature manager 130 may also use a second secret share to encrypt the same token and generate a second signature share. This process may be repeated for each available secret share to generate a set of signature shares from which a digital signature will be generated as described below.

At block 310, the signature manager 130 or some other module or component of the device 110 can obtain verification key data 126 representing the verification key to be used in verifying a digital signature generated using the signature shares generated using the secret shares.

At block 312, the signature manager 130 or some other module or component of the device 110 can generate a digital signature using the signature shares generated above at block 308. In some embodiments, signature shares may be combined to generate the digital signature. For example, signature shares may be multiplied or added together to obtain a digital signature, depending upon the digital signature thresholding scheme being used. Generally described, the signature shares may serve as input into a function that generates a digital signature using the signature shares. The output of the function may be an encrypted version of the token from which the signature shares were generated.

At block 314, the signature manager 130 or some other module or component of the device 110 can verify the digital signature using the verification key. In some embodiments, the signature manager 130 can decrypt the message, generated above at block 312, using a predefined decryption function and the verification key as the decryption key. If the decrypted message matches the original data item (or hash of the original data item), then the correct digital signature was determined. Otherwise, if the decrypted message does not match the original data item (or hash thereof), then an incorrect digital signature was determined.

At decision block 314, the signature manager 130 or some other module or component of the device 110 can determine whether the digital signature was verified above at block 314. If so, the process can proceed to block 318, where the function is authorized. For example, the software that is to be executed may be permitted to execute. If the signature manager 130 determines that the digital signature was not verified above at block 314, then the process may proceed to block 320, where the function is not authorized. For example, the software that is to be executed may not be permitted to execute.

At block 322 the process 300 can terminate.

### Threshold-Based Authorization Using Weighted Secret Shares

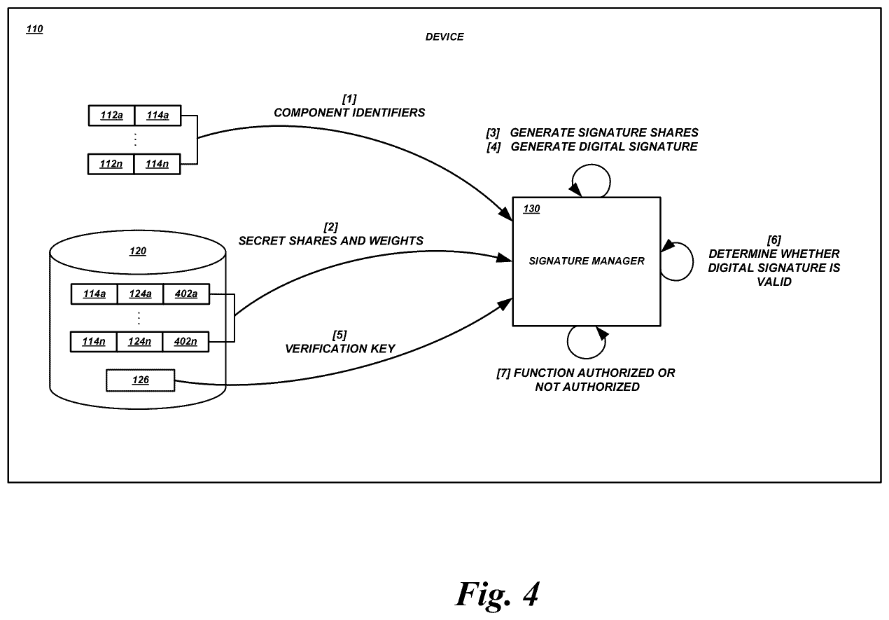

FIG. 4 is a block diagram of illustrative data flows and operations of a device 110 during threshold-based authorization using weighted secret shares. The displayed interactions may occur during authorization of a function, or authorization of operation of the device 110 itself. For example, a device 110 may be manufactured with an initial set of components 112. Some components 112 may be more important to the operation of the device 110, such that the device 110 is not permitted to operate without the items, or without particular versions of the items (e.g., original equipment manufacturer or “OEM” versions). Illustratively, the MCU of an infusion pump may be considered important, such that the pump is not permitted to operate without an OEM version. A battery that provides power to the pump when not connected to a wired power source may be considered less important, such that the pump is permitted to operate with a non-OEM version of the battery. However, if too many other parts are non-OEM in addition to the battery, then the pump may not be permitted to operate, even if “important” components like the MCU are present. To facilitate such treatment based on the characterized importance of the components, the individual components may be assigned weighting factors or given variable numbers of secret shares based on their characterized importance. When an authorization process is performed, the threshold amount of secret shares required may be a threshold amount of weighted secret shares based on the weighting factors or sets of secret shares assigned to each present component.

At [1], the signature manager 130 or some other module or component of the device 110 can obtain identifiers for the components that are present in the device 110. In some embodiments, the signature manager 130 can obtain identifiers 114 for all components 112 of the device 110, or for only certain components 112 (e.g., the n most important components, or the components specified for the function being authorized).

At [2], the signature manager 130 or some other module or component of the device 110 can obtain secret shares and weights associated with the component identifiers obtained at [1]. The weights and secret shares may be obtained from a data store 120, a separate data store in the signature manager 130, or some other module or opponent of the device 110. In some embodiments, the weights and secret shares may have been set and stored on the device 110 when the device 110 was manufactured. For example, the manufacturer may determine the weights according to a heuristic that prioritizes some components over other components by assigning higher weights (e.g., weights between 0.75 and 1.0) to components deemed more important, and lower weight (e.g., weights between 0.5 and 0.75) to components deemed less important. Share data 124a . . . 124n representing secret shares, and weight data 402a . . . 402n representing weights, may be stored in the data store 120 or some other module or component of the device 110. In some embodiments, rather than using different weights to represent the relative importance of the components, the components may instead be assigned a different number of secret shares depending upon their relative importance (e.g., more important components may be assigned more secret shares than less important components). For example, if a component has a weight of 5, then there may be 5 different secret shares allotted to the component, while another component with a weight of 1 may have only 1 share allotted. The example weighting methods described herein are illustrative only, and are not intended to be limiting. In some embodiments, other methods for using weighted secret shares may be used.

At [3], the signature manager 130 or some other module or component of the device 110 can generate digital signature shares, if possible, using the secret shares and weights. In some embodiments, the signature manager 130 may generate a different signature share using the same token and each of the different secret shares, as described in greater detail above.

At [4], the signature manager 130 or some other module or component of the device 110 can generate a digital signature using the signature shares determined above. In some embodiments, a predefined function may use the signature shares to generate the digital signature. The output of the function may be an encrypted version of the input value (or hashed input value) from which the signature shares were generated.

At [5], the signature manager 130 or some other module or component of the device 110 can obtain the verification key data 126 representing the verification key to be used in verifying the digital signature that was generated using the signature shares.

At [6], the signature manager 130 or some other module or component of the device 110 can verify the message using the verification key obtained above. In some embodiments, the signature manager 130 can decrypt the message using a predefined decryption function and the verification key as the decryption key. If the decrypted message matches the original data item (or hash of the original data item), then the correct digital signature was determined. Otherwise, if the decrypted message does not match the original data item (or hash thereof), then an incorrect digital signature was determined.

At [7], the signature manager 130 or some other module or component of the device 110 can determine whether the digital signature was verified above at block 314. If so, the function (or operation of the device 110 itself) is authorized. For example, the device 110 may be permitted to enter a ready state in which the device 110 may be used normally. If the signature manager 130 determines that the digital signature was not verified, then the function (or operation of the device 110 itself) is not authorized. For example, the device 110 may enter a blocked state in which normal operation is blocked.

### Threshold-Based Multi-Component Authorization

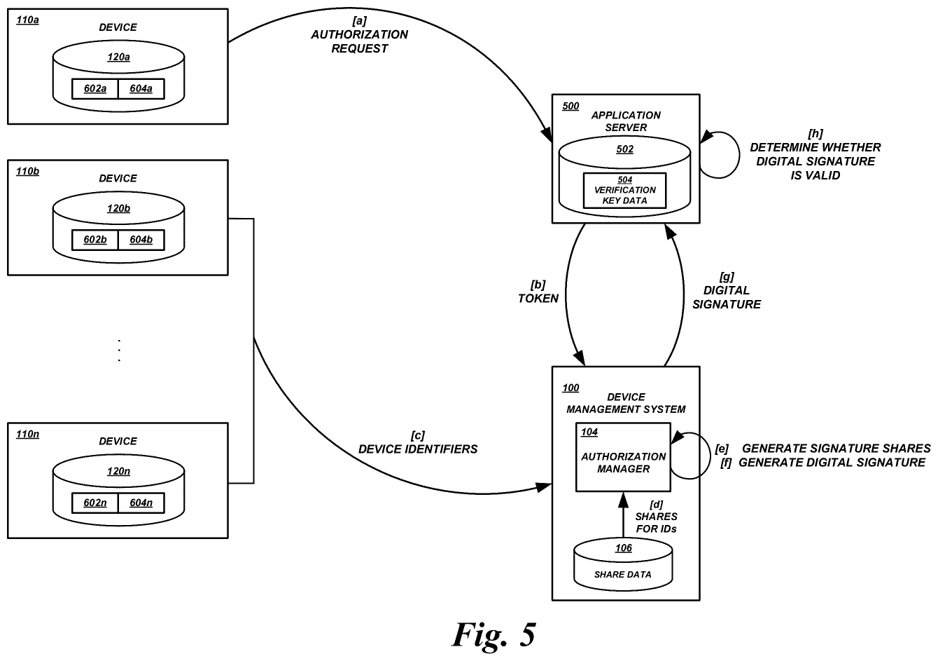

FIG. 5 is a block diagram of illustrative data flows and interactions between various devices 110a, 110b, . . . 110n, a device management system 110, and an application server 500 during threshold-based multi-component authorization (also referred to as “swarm” authentication). The displayed interactions may occur when a device, such as device 110a, connects to an application server 500 or otherwise attempts to perform a network operation. The application server 500 (or some other component of the network environment) may block the device 110a from connecting to the application server 500 or performing some other network function until the device 110a is authorized. The authorization process may require a threshold number of other devices 110b, . . . 110n to participate, actively or passively. By requiring the device 110a to be authorized using multiple other devices, the device 110a can be prevented from operating unless it is connected to a network with a threshold number of other devices. This prevents the use of a single device or small number of devices (e.g., devices that may be lost or stolen, or are otherwise being used outside of a multi-device network environment such as a hospital).

At [a], a device 110a attempts to connect to the application server 500 or perform some other network operation that requires the device 110a to first be authenticated. In some embodiments, the device 110a may be medical device, such as an infusion pump, and the application server 500 may be a related clinical system, such as an infusion control server. Illustratively, the infusion control server may instruct infusion pumps regarding the medication to be dispensed, the patient to be treated, the dosage to be infused, the security measures to be implemented, etc. Accordingly, the manufacturer of the infusion pumps may be interested in blocking operation of the infusion pumps (e.g., preventing the pumps from receiving medication dispensing information) unless and until the infusion pumps can first verify that they are part of a proper clinical environment, such as an environment with a threshold number of other such pumps.

At [b], the application server 500 may employ the services of the device management system 100 to authenticate the device 110a. The application server 500 may generate a token or other data to be signed by the device management system 100. The application server 500 may store or otherwise have access to a verification key that can be used to verify the signed message that is received from the device management system 100, as discussed in greater detail below. In some embodiments, the application server 500 may perform some or all of the functions described below as being performed by the device management system 100, or the device management system 100 may perform some or all of the function described as being performed by the application server 500. For example, the application server 500 and device management system 100 may be subsystems of a larger integrated system that includes one or more computing devices configured to perform the disclosed operations.

At [c], the device management system 100 can obtain identifiers and/or corresponding secret shares of the devices 110b . . . 110n that will participate in the multi-device authorization. For example, individual devices 110b . . . 110n can each provide their secret shares 604b . . . 604n to the device management system 100 when the devices 110b . . . 110n boot up, connect to the network environment, connect to the application server 500, are themselves authorized, or the like. As another example, the devices 110b . . . 110n can provide their identifiers 602b . . . 602n (e.g., serial numbers, internet protocol or “IP” addresses, media access control or “MAC” addresses, application-specific identifiers, etc.). The device management system 100 can use the identifiers at [d] to access secret shares corresponding to the devices. As a further example, the device management system 100 may retrieve secret shares and/or identifiers from the devices 110b . . . 110n in response to receiving a token and authentication request from the application server 500.

The secret shares may be assigned to the devices when the devices are manufactured, when the devices are implemented in the network environment, as the devices are authenticated using a multi-device authorization process, etc. In some embodiments, secret shares may be assigned to devices for periods of time, or secret shares may be revoked in response to the occurrence of particular events. For example, a device 110b may be assigned a secret share for a predetermined or dynamically determined period of time (e.g., 1 hour, 1 day, 1 year, 1 network communication session, etc.) after which the device 110b must be reassigned a share or have its secret share renewed (e.g., upon completion of a multi-device authorization process for the device 110b).

At [e], the authorization manager 104 or some other module or component of the device management system 100 can generate signature shares, if possible, using the secret shares, as discussed in greater detail above. In some embodiments, a predefined encryption function may use individual secret shares to encrypt the same data item, generating a set of signature shares. The data item may be the token or other data received from the application server 500. The data item may be encrypted directly, or it may be processed first (e.g., a hash may be generated using a hashing algorithm). This process may be repeated for each available secret share as described above.

At [f], the authorization manager 104 or some other module or component of the device management system 100 can generate a digital signature using the signature shares determined above. A valid digital signature may only be generated if a threshold number of secret shares, corresponding to a threshold number of devices 110a . . . 110n, are available at the time the authorization process is performed. If there are fewer than the threshold number of secret shares, then a valid digital signature may be impossible or computationally infeasible to determine. In this instance, the authorization process may be aborted, and/or an error message or other failure message may be returned to the application server 500.

At [g], the authorization manager 104 or some other module or component of the device manager 100 can send the digital signature (e.g., the encrypted token) to the application server 500.

At [h], the application server 500 can access verification key data 504 representing the verification key, and decrypt the message using a predefined decryption function and the verification key as the decryption key. If the decrypted message matches the original token that was sent to the device manager 100 (or data derived therefrom, such as a hash), then the correct digital signature was determined. In this instance, the device 110a may be considered authorized because at least a threshold number of other devices participated in the authorization process. Otherwise, if the decrypted message does not match the original data item (or hash thereof), then an incorrect digital signature was determined. In this instance, the application server 500 may block the device 110a from performing the function that the device 110a is attempting to perform because the device 110a has not been authorized. This may occur when fewer than a threshold number of devices participate in the process, such as when the device 110 is being used outside of an intended clinical environment (e.g., the device is lost, stolen, or sold).

### Threshold-Based Security Enforcement Using Weighted Secret Shares

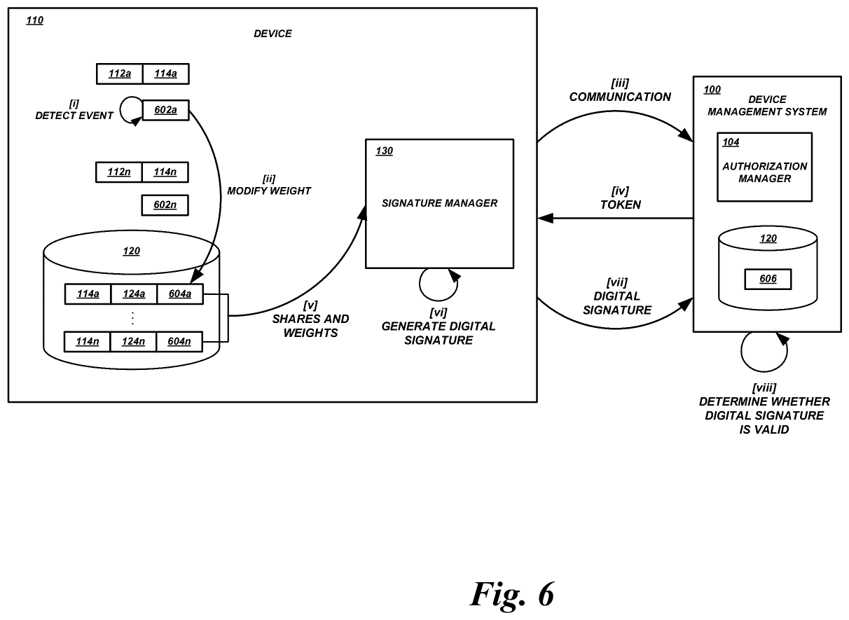

FIG. 6 is a block diagram of illustrative data flows and interactions between a device 110 and a device management system 100 during threshold-based security enforcement. In threshold-based security enforcement, different components of a multi-component system can participate in identifying security risks and enforcing security policies. For example, different components 112 of a single device 110 may participate, different devices 110 of a distributed system may participate, etc. Advantageously, the individual components may each be assigned a weight. As the components (or separate security modules with which the components are associated) detect the occurrence of security events that impact the security of the system, the components can adjust their weighting factors to reflect the impact. For example, if one security monitoring module (also referred to as a security monitor or security module) determines that a security risk has occurred or is likely to occur, the security module may reduce the weighting factor for the component to which the security module is assigned. The secret shares and weighting factors of the various components are used to authorize system functions. The authorization process will fail if enough components have provided lower weights such that a threshold amount of weighted secret shares is not used during the authorization process.

The displayed interactions may occur when a device 110 connects to the device management system 100 or otherwise attempts to perform a network operation. In some embodiments, threshold-based security enforcement may be used when the device 110 communicates with an application server, such as the application server 500 described above.

At [i], a security module 504 associated with a particular component 112a of the device 100 may determine that a security event has occurred. For example, the security module 602a may detect security events such as failed login attempts, medication or dosing alerts, security override events, repeated pings, network scanning activity, denial of service conditions, other alerts or errors, and the like. In some embodiments, the device 110 may be a medical device, such as an infusion pump. In this instance, the component 112a may be a component of the infusion pump, such as an MCU, and the security module 602a may be assigned to or integrated with the MCU. The security module 602a may detect security events such as invalid attempts to infuse medication, alerts indicating an attempt to infuse an unsafe dose, or the like.

At [ii], the security module 602a may modify the weight 604a based on the security event(s) that the security module 602a has detected. The degree to which the weight 604a is adjusted may be incremental, or it may be dynamically determined based on the particular event(s) detected. For example, the weight 604a may have a starting or default value (e.g., 1.0) when the device 110 begins operation. The security module 602a may be configured to lower the weight 604a by an incremental amount for each security risk that is detected (e.g., lower the weight by 0.05 for each failed login attempt, each medication dose alarm, etc.). As another example, the security module 602a may be configured to lower the weight by a varying amount depending upon the particular security risk identified (e.g., lower the weight by 0.1 after three failed login attempts, lower the weight by 0.2 after two medication dose alarms, etc.).

In some embodiments, the security module 602a may increase the weight 604a in response to certain events, after the passage of a predetermined or dynamically determined period of time, etc. For example, the security module 602a may increase the weight 604a after a period of time (e.g., 10 minutes, 1 hour, etc.) has passed without a negative security event occurring. As another example, the security module 602a may reset the weight 604a to its default value in response to an administrator logging into the system. In some embodiments, the security module 602a may only adjust a weight 604a between maximum and minimum values (e.g., the weight may not be permitted to fall below 0.0 or rise above 1.0).

The example security events, weighting factor values, and adjustment rules described herein are illustrative only, and are not intended to be limiting. In some embodiments, fewer, additional, and/or alternative events, weighting factor values, and adjustment rules may be used.

At [iii], the device 110 can attempt to perform a particular function, such as connect to the device management system 100 or perform some other network operation that requires the security status of the device 110 to first be validated. For example, if the device 110 is a medical device such as an infusion pump, the device may attempt to infuse medication, connect to a network server to obtain data regarding medications, doses, or patients, etc. Accordingly, the manufacturer of infusion pumps may be interested in blocking operation of the infusion pumps (e.g., preventing the pumps from receiving medication dispensing information) unless and until the security status of the infusion pumps can first be validated.

At [iv], the device management system 100 may generate a token or other data to be signed by the device 110. The device management system 100 may store or otherwise have access to a verification key that can be used to verify the signed message that is received from the device 110, as discussed in greater detail below.

At [v], the signature manager 130 or some other module or component of the device 110 can obtain identifiers and corresponding secret shares and weights for the components 112a . . . 112n. For example, the verification manager 130 may determine the identifiers 114a . . . 114n of the components 112a . . . 112n. The signature manager 130 may then obtain the corresponding secret shares 114a . . . 114n and weights 604a . . . 604n from the data store 120.

At [vi], the signature manager 130 or some other module or component of the device 110 can generate signature shares using the secret shares 114a . . . 114n and weights 504a . . . 504n, as discussed in greater detail above. A digital signature may then be generated using the signature shares. If the amount of weighted secret shares fails to satisfy the threshold amount, then a valid digital signature may be impossible or computationally infeasible to determine. In this instance, the authorization process may be aborted, and/or an error message or other failure message may be returned.

If a threshold number of signature shares are generated, the signature manager 130 can then generate the digital signature, as described in greater detail above.

At [vii], the device 110 can send the signed message (e.g., the encrypted token) to the device management system 100.

At [viii], the device management system 100 can obtain the verification key data 606 representing the verification key, and decrypt the message using a predefined decryption function and the verification key as the decryption key. If the decrypted message matches the original token that was sent to the device management system 100 (or data derived therefrom, such as a hash), then the correct digital signature was determined. In this instance, the device 110 may be considered to be in a secure state because at least a threshold amount of weighted secret shares, as maintained by the various components of the device 110, were available for the authorization process. Otherwise, if the decrypted message does not match the original data item (or hash thereof), then an incorrect digital signature was determined. In this instance, the device management system 100 may block the device 110 from communicating with the device management system 100, other network systems (e.g., application server 500), performing particular operations, etc.

### OTHER CONSIDERATIONS

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a computer processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A computer processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. An infusion pump comprising:
a motor control unit configured to control infusion of medication, wherein the motor control unit is associated with a first component identifier;
a battery configured to power the infusion pump, wherein the battery is associated with a second component identifier;
a computer processor programmed with executable instructions, wherein the computer processor is associated with a third component identifier; and
a data store storing:
verification key data representing a verification key; and
share data representing a plurality of secret shares for generating a digital signature, wherein a first secret share of the plurality of secret shares is associated with the first component identifier, wherein a second secret share of the plurality of secret shares is associated with the second component identifier, and wherein a third secret share of the plurality of secret shares is associated with the third component identifier;
wherein the computer processor is programmed by the executable instructions to at least:
determine that a command has been issued for execution of software that controls a function of the infusion pump;
determine a plurality of component identifiers, wherein individual component identifiers of the plurality of component identifiers correspond to individual components of the infusion pump present at a time the command is issued;
load at least a subset of the plurality of secret shares based at least partly on the plurality of component identifiers;
generate a plurality of signature shares using the subset of the plurality of secret shares, wherein a threshold number of secret shares is required in order to generate a threshold number of signature shares;
generate the digital signature using the plurality of signature shares;
verify the digital signature using the verification key; and
authorize execution of the software.

2. The infusion pump of claim 1, wherein the data store further stores a plurality of weights, wherein individual weights of the plurality of weights are associated with individual secret shares of the plurality of secret shares, and wherein the threshold number of secret shares comprises a threshold amount of weighted shares.

3. The infusion pump of claim 2, further comprising a security monitor configured to at least:
detect occurrence of a security event; and
modify a value of a weight based at least partly on the security event, wherein the weight is associated with a component of the infusion pump, and wherein the security monitor is associated with the component of the infusion pump.

4. The infusion pump of claim 3, wherein the security event comprises one of: a failed login attempt, a medication alert, a security override attempt, a repeated ping, network scanning activity, a denial of service event, or an error.

5. A computer-implemented method for authorizing execution of infusion system commands, the computer-implemented method comprising, as performed by one or more computing devices configured to execute specific instructions:
determining a plurality of component identifiers, wherein each component identifier comprises an identifier of a corresponding component of an infusion system;
determining, for each component identifier of the plurality of component identifiers, a corresponding secret share for generating a digital signature;
storing the plurality of component identifiers and corresponding secret shares;
storing a verification key associated with the secret shares;
receiving a system command for execution; and
initiating a pre-execution authorization procedure comprising:
determining at least a subset of component identifiers of the plurality of component identifiers, wherein the subset of component identifiers corresponds to components of the infusion system present during the pre-execution authorization procedure;
loading at least a subset of secret shares, wherein each secret share of the subset of secret shares corresponds to a component identifier of the subset of component identifiers;
generating the digital signature using the subset of secret shares, wherein a threshold amount of secret shares are required in order to generate the digital signature;
verifying the digital signature using the verification key; and
authorizing execution of the system command.

6. The computer-implemented method of claim 5, further comprising determining, for each secret share, a corresponding weighting factor, wherein a threshold amount of weighted secret shares are required in order to generate the digital signature.

7. The computer-implemented method of claim 6, further comprising:
detecting occurrence of a security event; and
modifying a value of a weighting factor based at least partly on the security event.

8. The computer-implemented method of claim 7, wherein detecting occurrence of the security event comprises detecting one of: a failed login attempt, a medication alert, a security override attempt, a repeated ping, network scanning activity, a denial of service event, or an error.

9. The computer-implemented method of claim 5, further comprising executing the system command in response to authorizing execution of the system command, wherein executing the system command comprises one of: executing license software, dispensing medication, or communicating with a network server.

10. The computer-implemented method of claim 5, wherein storing a secret share comprises storing the secret share in a secure storage location associated with a corresponding component of the infusion system.

11. The computer-implemented method of claim 5, wherein storing a secret share comprises storing an obfuscated version of the secret share.

12. A system comprising:
a plurality of medical devices;
an authorization manager; and
an application server comprising one or more computing devices, wherein the application server is configured to at least:
receive, from a first device of the plurality of medical devices, a communication regarding operation of the first medical device;
determine to initiate an authorization process to authorize the first medical device prior to responding to the communication;
send a token to the authorization manager;
receive a digital signature from the authorization manager;
verify the digital signature using a verification key and the token;
determine, based on verifying the digital signature, that the first medical device is authorized to operate; and
respond to the communication; and
wherein the authorization manager comprises one or more computing devices and is configured to at least:
receive the token from the application server;
identify at least a subset of the plurality of medical devices based at least partly the subset of the plurality of medical devices being in communication with the authorization manager;
access secret share data associated with at least the subset of the plurality of medical devices, wherein secret share data associated with a first medical device of the subset represents a secret share for generating a digital signature, and wherein a threshold number of secret shares are required in order to generate the digital signature;
generate the digital signature using the secret share data and the token; and
send the digital signature to the application server.

13. The system of claim 12, wherein the application server being configured to verify the digital signature comprises the application server being configured to at least:
generate a decrypted message using the digital signature and the verification key, wherein the verification key comprises a decryption key for a decryption function; and
determine that the decrypted message comprises data representing the token.

14. The system of claim 12, wherein the authorization manager being configured to generate the digital signature comprises the authorization manager being configured to encrypt the token using a secret share as an encryption key for an encryption function.

15. The system of claim 12, wherein the authorization manager being configured to generate the digital signature comprises the authorization being configured to at least:
generate a threshold number of signature shares using the threshold number of secret shares; and
generate the digital signature using the threshold number of signature shares.
